(19) United States
(12) Patent Application Publication
(10) Pub. No.: US 2011/0229553 A1
(43) Pub. Date: Sep. 22, 2011

(54) Plant Extract with Pharmacological Profile for Treating or Preventing Diseases, Dysfunctions and Disorders of the Central Nervous System, Neurodegenerative Disorders and Sequel from Vascular Dementia

(76) Inventors: Elaine Elisabetsky, Porto Alegre - RS (BR); Domingos Sávio Nunes, Ponta Grossa - PR (BR); Adriana Lourenco Da Silva, Pelotas - RS (BR); Ângelo Luis Stapassoli Piato, Caxias do Sul - RS (BR); Carlos Alexandre Netto, Porto Alegre - RS (BR); Ionara Rodrigues Siqueira, Porto Alegre - RS (BR); Micheli Figueiró, Santa Cruz do Sul - RS (BR); Matilde Achaval Elena, Porto Alegre - RS (BR); Lisiane Porciuncula, Porto Alegre - RS (BR)

(21) Appl. No.: 13/009,016

(22) Filed: Jan. 19, 2011

Related U.S. Application Data
(60) Provisional application No. 61/297,442, filed on Jan. 22, 2010.

(51) Int. Cl. A61K 36/185; A61K 9/127; A61P 25/28; A61P 25/16; A61P 25/24

(52) U.S. Cl. 424/450; 424/769

(57) ABSTRACT

Plant extracts for pharmaceutical compositions as acetylcholinesterase inhibitors useful as neuroprotectors, to manage depressive states and cognitive deficits of diverse etiologies, and for the treatment of neurodegenerative conditions, such as Alzheimer's and Parkinson's diseases, and the sequel from ischemic events.

### FIELD OF THE INVENTION

The present application is in the field of plant extracts for pharmaceutical compositions as acetylcholinesterase inhibitors useful as neuroprotectors, to manage depressive states and cognitive deficits of diverse etiologies, and for the treatment of neurodegenerative conditions, such as Alzheimer and Parkinson diseases, and the sequel from ischemic events.

### BACKGROUND OF THE INVENTION

The differentiation of a normal healthy ageing and pathologic conditions common to the elderly is not always clear-cut. Ageing is, essentially, a degenerative process that culminates with neural death. Chronic neurodegenerative diseases are characterized by a progressive and irreversible neuronal loss in specific brain areas, as a result of neuronal injury consequent to a complex interaction of genetic and environmental factors.

Cognitive deficits are frequently associated with ageing and a core sign in dementias, with a global prevalence predictable to increase with the increasing life expectancy. Dementias affect approximately 5% of older people at 65, and 20% of those over 80 years old Among most common chronic-degenerative diseases are the Parkinson Disease (PD), affecting 1% of population over 65 years of age, and the Alzheimer Disease (AD) which became the commonest form of dementia in the elderly, affecting 2% of this group in developing countries. The current treatment for Alzheimer disease is based on acetylcholinesterase inhibitors (AChEIs), used for the mild and moderate stages of the disease. The ideal Ache would be well tolerated, of convenient administration, would induce a selective and sustained inhibition in the brain, besides showing selectivity to the isoforms of the enzyme of most relevance in AD, especially in the cortex and hippocampus. AChEIs such as this are not currently available.

Vascular dementia, resulting from small and recurring brain infarcts, is responsible for approximately 20% of all dementias pathologically confirmed. The prevalence of vascular dementia in individuals older than 64 years of age is estimated to be 1.0% whereas that of AD in 2.4%. In fact, vascular dysfunction responsible for changes in small vessels and hypo perfusion may precede the dementia in AD, strongly suggesting that cerebral ischemia has an important role in the majority of degenerative dementias. The world faces a prevalence of dementia of an epidemic nature associated with the increasing life spam, and brain ischemia may be one of the major contributing factors. The cost of cerebral ischemia, and of the associated pharmaceutical market, may be assessed by the following numbers: cerebral-vascular diseases are responsible for 5.4 millions of death/year (10% of the total), consuming £21 billion or approximately 3% of the health system costs in the European community in 2003; this costs raised to £34 billion if informal care and productivity losses are included; the anticipated costs of cerebro-vascular accidents in the US economy between 2005 and 2050 is U$ 2.2 trillion). Although historically seen as an inevitable consequence of ageing, it is now well accepted that the consequences of cerebral ischemia are prone to both prevention and treatment.

Relevant to this application, it has been shown that a combination of memantine (NMDA antagonist) and donezepil (AChEI) has a better outcome than any of the drugs given alone in the treatment of both vascular dementia (moderate and severe) and the latter stages of AD (Rossom, R., Adityanjee, Dysken, M., 2004. Efficacy and tolerability of memantine in the treatment of dementia. Am J Geriatr Pharmacother, 2: 303-312.). In fact, several approaches indicate that multi or bi-functional compounds may result in higher effectiveness as neuroprotective agents than those with a single mechanism of action. It has been argued that the historical difficulty for the development of better psychiatric drugs was the valorization of few targets as pharmacologic mechanism of actions and the unlikely belief in a single abnormal molecule as cause of complex illnesses. This notion is perfectly compatible with the demonstration that neurodegenerative phenomena are multifactorial in nature, determining a renewed interest in plant drugs having more than an active ingredient, and/or compounds with innovative and multiple mechanisms of action, and/or by the synergic interaction of these various active compounds.

Depression is another mental disturb common in the elderly, in general considered a chronic, recurrent, potentially fatal pathology that affects 20% of the global population. Our data show a clear antidepressant-like effect of the extract, demonstrated in three animal models, in a dose range lower than that presenting promnesic properties. The data show that the antidepressant activity depends on norepinephrine, and possibly not of serotonin, as well as involving the participation of dopamine D1 receptors and β adrenergic. Normalization of the hypothalamic-pituitary-adrenal axis (HPA) is related to the success of antidepressant treatment, and the data indicate that the compounds may normalize the HPA axis in an animal model of depression associated with repetitive stress. (Roth, B. L., Sheffler, D. J., Kroeze, W. K., 2004. Magic shotguns versus Magic bullets: selectively non-selective drugs for mood disorders and schizophrenia. Nat Rev Drug Discov, 3: 353-359). Given that depression includes cognitive deficits and can either trigger or influence the progression of neurodegenerative diseases, the antidepressive properties of the compounds add to its overall therapeutic value in neurodegenerative diseases.

The medical and scientific literatures identify physical exercise as a preventive measure not only to cardiovascular diseases, but also cancer, depression and neurodegenerative diseases.

Ptychopetalum olacoides Bentham (PO) (Olacaceae) is a plant most commonly used as a “nerve tonic” in the Amazon, now also found in herbals in Brazil, Europe and USA. “Nerve tonic”, “stimulating nerves,” or simply “tonics” are found in many traditional medical systems, commonly used by the elderly or those convalescent from disease in general and specifically from those that affect the central nervous system (such as stroke lack of concentration, memory lapses), and/or during periods of intense physical or mental stress. In addition to articles of the medical literature specifically, the patent literature also has a number of publications devoted to herbal medicines using Ptychopetalum olacoides Bentham (PO) (Olacaceae).

JP9235237A (1997) describes the invention relating to the use of a composition comprised of Muirapuama and Cordyceps sinensis Sacc. The composition is allegedly capable to enhancing functions and effectively acting on a state of deteriorated physical strength due to a stress. The Muirapuama can be used as an extract and the daily dose thereof for an adult is about 10-5000 mg expressed in terms of the amount of the raw crude drug. The Cordyceps sinensis Sacc. can be used as an extract or a fluid extract and the dose thereof administered is about 50-1000 mg.

JP2000119187A (2000) describes the invention relating to the use of a composition obtained by formulating Muirapuama or its essence. The effective daily dose for an adult is preferably about 10-500 mg expressed in terms of the amount of the raw crude drug. Furthermore, a water-soluble vitamin, a xanthin derivative, a crude drug, an excipient, a pH adjustor, etc., may be formulated.

WO0072861A1 (2000) and U.S. Pat. No. 6,746,695 B1 (2004) describe methods for extracting and purifying bioactive substances from various plants and herbs. More specifically the invention relates to methods of extracting and separating bioactive substances from various plants and herbs, such as Kava root, Byrsonima species, Aesculus californica, Crataegus mexicana, Simmondsia chinensis, Pfaffia species, Alternanthera repens, Bursera species, Turnera species, Perezia species, Heimia salicifolia, Psidium species, Enterlobium species, Ptychopetalum olacoides, Liriosma ovata, and Chaunochiton kappleri, using supercritical fluid extraction and/or fluorocarbon solvent extract. The invention further relates to separation of bioactive substances contained in extracts using packed column supercritical fluid chromatography or HPLC, where dense gas with or without modifiers is the mobile phase. The invention also relates to pharmaceutical preparations and dietary supplements which may be prepared with the extracted bioactive substances and use of such pharmaceutical preparations and dietary supplements to treat various human aliments.

Brazilian PI0102185-0 (2001) describes the use of the product comprising extract as an antioxidant or as a cerebral vasodilator agent, pharmaceutical composition comprising such product for the prophylaxis or treatment of vascular disorders and disturbances caused by the inappropriate presence of free radicals, the method for prophylaxis or treatment of cerebrovascular disorders and disorders caused by the inappropriate presence of free radicals using the product and use of that product to produce a pharmaceutical composition for prophylaxis or treatment of vascular disorders and disturbances caused by the inappropriate presence of free radicals. The invention addresses the use of a product of plant extracts including the species Trichilia sp, Paullinia cupana (Sapindaceae), Ptychopetalum olacoides (Olacaceae) and Zingiber officinale (Zingiberaceae).

Brazilian P10102184-2 (2001) describes the use of extract as an antidepressant and anxiety disorders, pharmaceutical composition comprising such product for the treatment or prevention of depression and/or anxiety disorders, the method for treatment or prevention of depression and/or anxiety disorders using the product and use of that product to produce a pharmaceutical composition for treatment or prevention of depression and/or anxiety disorders. The invention is the use of an extract product comprising the inlet species Trichilia sp (preferably from Trichilia catigua) Paullinia cupana (Sapindaceae), Ptychopetalum olacoides (Olacaceae) and Zingiber officinale (Zingiberaceae).

Brazilian P10102186-9 (2001) describes use of the product comprising extract as agent, pharmaceutical composition comprising such product for the treatment or prevention of thromboembolic disorders, the method for treatment of thromboembolic disorders using the product and use of this product for production of a pharmaceutical composition for treatment or prevention of thromboembolic disorders. The invention is the use of a product extracts inlet including the species Trichilia sp (preferably the kind catigua) Paullinia cupana (Sapindaceae), Ptychopetalum olacoides (oliacaceae) and Zingiber officinale (Zingiberaceae).

Brazilian PI0307647-4 A2 (2003) describes the invention of the extraction process of the chemical and pharmaceutical compositions. This paper describes the use of ethanol extracts of plants of the family Olacaceae as a chemical and/or pharmaceutical compositions for the prevention and treatment of chronic degenerative disorders of the central nervous system based on verification testing of biological activity for therapeutic purposes desired. The ethanol extracts endowed with biological activity are obtained using ethyl alcohol/water in proportions varying between 50 and 95% ethyl alcohol, characterized by the presence of a chemical marker substance or guide called pov-2. It also described a process of obtaining and identification of the substance guide pov-2 from plants of the family Olacaceae.

U.S. Pat. No. 6,746,695 (2004) describes methods of extracting and purifying bioactive substances from various plants and herbs. More specifically the invention relates to methods of extracting and separating bioactive substances from various plants and herbs, such as Kava root, Byrsonima species, Aesculus californica, Crataegus mexicana, Simmondsia chinensis, Pfaffia species, Alternanthera repens, Bursera species, Turnera species, Perezia species, Heimia salicifolia, Psidium species, Enterlobium species, Ptychopetalum olacoides, Liriosma ovata and Chaunochiton kappleri, using supercritical fluid extraction and/or fluorocarbon solvent extract. The invention further relates to separation of bioactive substances contained in extracts using packed column supercritical fluid chromatography or HPLC where dense gas with or without modifiers is the mobile phase. The invention also relates to pharmaceutical preparations and dietary supplements which may be prepared with the extracted bioactive substances and use of such pharmaceutical preparations and dietary supplements to treat various human ailments. Another embodiment of the invention is directed to formula and compositions comprising a combination of extracted phytochemicals from Turnera species and Pfaffia species, with or without muira puama (a crude drug derived from various species including Ptychopetalum olacoides, Liriosma ovata, and Chaunochiton kappleri for use as a health tonic and to support sexual function.

JP2005350391A (2005) describes the use of one or two or more species selected from the group consisting of plants of the genus Picrorhiza, plants of the Apocynum L., Catharanthus roseus (L.) Don, or the like, plants of the genus Iris, plants of the genus Rubus, plants of the genus Gossypium, plants of the genus Cynamchum, plant of the genus Tylophora, plants of the family Cactacea, plant of the genus Ceratostigma, plants of the genus Hyoscyamus, Hercampure; Gentianella alborosea (Gilg), Hernandia peltata, Ptychopetalum olacoides and pyroligneous acid are applied as an active ingredient. Thereby, neurocyte apoptosis by the Alzheimer's disease can be suppressed to carry out the prophylaxis of the Alzheimer's disease, suppress the progression of the Alzheimer's disease and treat the Alzheimer's disease.

Brazilian PI0605812-4 A2 (2006) describes the use of combination of extracts from pfaffia (Pfaffia sp.), maripuama (Ptychopetalum olacoides) and white lily (Lilium candidum) in improvement of specific skin changes; in particular the use of a mixture of concentrated extracts of pfaffia (Pfaffia sp.) puama (Ptychopetalum olacoides) and lily (Lilium candidum), presented as a hidroglicolic extract pure or mixed with other extracts and/or ingredients in cosmetic preparations and pharmaceuticals for treatment and improvement of the physiological and aesthetic of the region around the eyes, as dark circles or hyperpigmentation, edema or swelling, fat pads and the formation of fine wrinkles to the around the eyes, where this effect is achieved through mechanisms of action that lead to anti-inflammatory activities, decongestants, draining, lipolytic and restorative.

Applicant has shown that Ptychopetalum olacoides contains bioactive compounds with central action. dissertation of Ionara Rodrigues Siqueira (Contribution to the ethnopharmacology of Ptychopetalum olacoides Bentham: psychopharmacological properties. Masters Thesis, Masters in Biological Sciences—Physiology, Presented in November 1997, UFRGS, Brazil) and the following articles: Elisabetsky, E., Smith, I. R., 1998. Is there a psychopharmacological meaning for traditional tonics? IN: Prendergast H. D., Etkin N., Harris D. R. Houghton P. J (eds), Plants for Food and Medicine, 373-385. Royal Botanical Gardens, Kew and Siqueira, I. R., Lara, D. R., Silva, D., Gaieski, F. S., Nunes, D. S., Elisabetsky, E., 1999. Psychopharmacological properties of Ptychopetalum olacoides BENTHAM (Olacacea). Pharm Biol, 36 (5): 327-334).

Despite the range of indications (referred to but not specified or associated with biological data to substantiate the indications) and procedures for the extraction of bioactive compounds (generally referred to but not named) from Ptychopetalum reported in the literature, there is no mention or suggestion of the psychopharmacological properties and neurochemical data described bellow for P. olacoides extracts or that have the following pharmacological properties useful in preventing and/or treating degenerative diseases of the central nervous system. Such uses are described and claimed in this application.

### SUMMARY OF THE INVENTION

The plant extract and composition of the present application is related to innovative mechanisms of action in line with the latest approaches to development of antidepressant drugs. The extract increases endurance, a pattern likely to result from an altered and more effective energy consumption (glycogen sparing along with increased fatty acid burning), and protection from muscle damage (decreased CK and LDH during exercise). These properties add to the overall therapeutic value of this extract in treating and or preventing neurodegenerative diseases.

The present application seeks to provide an ethanolic extract of Ptychopetalum olacoides obtained from a source selected from a group consisting of: stems, barks, leaves, roots and combinations thereof.

The present application also seeks to provide a pharmaceutical composition comprising: (a) an effective amount of an extract of Ptychopetalum olacoides, and (b) pharmaceutically acceptable excipients. The pharmaceutical composition comprising from 0.001% to 99% of extract of Ptychopetalum olacoides.

The present application also seeks to provide a method for treating or preventing diseases, dysfunctions and disorders of the central nervous system; neurodegenerative disorders and sequel from vascular dementia in a patient by administering an effective amount of an extract of Ptychopetalum olacoides.

### BRIEF DESCRIPTION OF THE DRAWINGS

FIG. 1 shows the effect of Ptychopetalum olacoides extract (POEE) and eserine on G1 (A) and G4 (B) AChE isoforms in mouse hippocampus. SAL=saline; ESE=Eserine. All assays were performed in triplicate for five separate experiments. Each value represents mean±S.E.M. *P<0.05 vs. control (saline and DMSO).

FIG. 2 shows the effect of Ptychopetalum olacoides extract (POEE) and eserine on G1 (A) and G4 (B) AChE isoforms in mouse frontal cortex. SAL=saline; ESE=Eserine. All assays were performed in triplicate for five separate experiments. Each value represents mean±S.E.M. *P<0.05 vs. control (saline and DMSO).

FIG. 3 shows the effect of Ptychopetalum olacoides extract (POEE) and eserine on G1 (A) and G4 (B) AChE isoforms in mouse striatum. SAL=saline; ESE=Eserine. All assays were performed in triplicate for five separate experiments. Each value represents mean±S.E.M. *P<0.05 vs. control (saline and DMSO).

FIG. 4 shows the Lineweaver-Burk representation of G1 AChE (A) and G4 AChE (B) inhibition by Ptychopetalum olacoides extract (POEE) in the hippocampus with acetylthiocholine as substrate. Double reciprocal plot was constructed by plotting 1/V against 1/S analyzed over a range of substrate concentrations (0.01-0.075 mM) in the absence and in the presence of Ptychopetalum olacoides extract (POEE) (30, 100, 300 and 1000 μg/mL). The plot represents the means of five experiments (n=5).

FIG. 5 shows the effects of Ptychopetalum olacoides extract (POEE) on AChE activity in mice hippocampus CA1 (A), CA3 (B) and striatum (C). SAL=saline; GALA=Galanthamine. Optical density (OD) is expressed in pixels. Data are presented as means±S.E.M. (n=5). *P<0.05 vs. control (DMSO).

FIG. 6 shows the effects of Ptychopetalum olacoides extract (POEE) on AChE activity in mice hippocampus (CA1 and CA3) and striatum.

FIG. 7 shows the effect of Ptychopetalum olacoides extract (POEE) ex vivo on G1 (A) and G4 (B) AChE isoforms in mouse hippocampus. SAL=saline; GALA=Galanthamine. All assays were performed in triplicate. Each value represents mean±S.E.M. *P<0.05 vs. control (DMSO).

FIG. 8 shows the effect of Ptychopetalum olacoides extract (POEE) ex vivo on G1 (A) and G4 (B) AChE isoforms in mouse frontal cortex. SAL=saline; GALA=Galanthamine. All assays were performed in triplicate. Each value represents mean±S.E.M. *P<0.05 vs. control (DMSO).

FIG. 9 shows the effect of Ptychopetalum olacoides extract (POEE) ex vivo on G1 (A) and G4 (B) AChE isoforms in mouse striatum. SAL=saline; GALA=Galanthamine. All assays were performed in triplicate. Each value represents mean±S.E.M. *P<0.05 vs. control (DMSO).

FIG. 10 shows the western blotting analysis for AChE immunocontent in total membranes from the mice hippocampus (A), and frontal cortex (B). Bands of the equivalent molecular weights (65 kDa for AChE) are illustrated on the top of the each histogram where bars indicate the bands quantifications by scanned autoradiographic films. Density is expressed as means±S.E.M. of five samples of whole hippocampus and frontal cortex for each treatment group. *P<0.05, vs. control (saline).

FIG. 11 shows the western blotting analysis for AChE immunocontent in synaptosomal fractions from the mice hippocampus (A) and frontal cortex (B). Bands of the equivalent molecular weights (65 kDa for AChE) are illustrated on the top of the each histogram where bars indicate the bands quantifications by scanned autoradiographic films. Density is expressed as means±S.E.M. of five synaptosomal samples hippocampus and frontal cortex for each treatment group. *P<0.05, vs. control (saline).

FIG. 12 shows the effect of Ptychopetalum olacoides extract (POEE) 800 mg/kg administrated for 14 days in adult mice on step-down inhibitory avoidance task (LTM, 24 h training-test interval). DMSO=dimethyl sulphoxide 20%; Sal=saline; Aβ₁₋₄₂=β-amyloid (1-42) peptide fragment; PO=standardized ethanol extract of Marapuama (N=12/group). Each column represents latencies (s) median (interquartile ranges) of training (light columns) or test (gray columns) latencies. ^{##}p<0.05 test×training latencies for each treatment, Wilcoxon. **p<0.05× controls (PBS+Sal) test latencies, Mann-Whitney/Kruskal-Wallis; ^{$}p<0.05 for Aβ₁₋₄₂+Sal×Aβ₁₋₄₂+PO test latencies, Wilcoxon.

FIG. 13 shows the effects of Ptychopetalum olacoides extract (POEE) on spontaneous locomotor activity: (A) exploration (first 3 min) and (B) locomotion (final 5 min). Each column represents the mean±SEM. N=12. ANOVA/Duncan's test.

FIG. 14 shows the brain-derived neurotrophic factor (BDNF) levels in mice hippocampus. Results are expressed as mean±SEM. N=5 per group. ANOVA/Duncan's test.

FIG. 15 shows the effects of Ptychopetalum olacoides extract (POEE) (200 mg/kg) and apomorphine 3 mg/kg (APO) on MPTP-induced tremors in C57BL/6 mice. The intensity of tremors was scored 0-5 by independent observers immediately after the second dose of MPTP. Evaluation was done every 3 min for a period of 45 min. N=5, mean±SEM, *P≦0.05 or # P≦0.01 vs. control, Kruskai Wallis/Mann Whitney.

FIG. 16 shows the effects of Ptychopetalum olacoides extract (POEE) (25 or 50 mg-kg) and apomorphine 3 mg/kg (APO) on BALB/c mice MPTP induced tremor. The intensity of tremors was scored as 0-5 by independent observers immediately after the second dose of MPTP. Evaluation was done every 3 min for a period of 45 rain. N=6, mean±SEM, *P≦0.05 or # P≦0.01 vs. control, Kruskal Wallis/Mann Whitney.

FIG. 17 shows the effects of Ptychopetalum olacoides extract (POEE) (25 and 50 mg/kg) and apomorphine 3 mg/kg (APO) on BALB/c mice MPTP-induced catalepsy. Assessment of catalepsy was undertaken 3 h after the treatment of MPTP. Results given are mean±S.E.M (five times for each animal), *P≦0.05× controls sal×sal or # P≦0.1× controls MPTP. Kruskal Wallis/Mann Whitney. n=6.

FIG. 18 shows the effects of Ptychopetalum olacoides extract (POEE) (25 and 50 mg/kg) and apomorphine 3 mg/kg (APO) on BALB/c mice MPTP-induced akinesia. Assessment of akinesia was undertaken 4 h after the treatment of MPTP. Results given are mean±S.E.M (five times for each animal), *P≦0.05× controls sal×sal or # P≦0.01× controls MPTP. Kruskal Wallis/Mann Whitney. n=6.

FIG. 19 shows the effect of different doses of Ptychopetalum olacoides extract (POEE) (25 and 50 mg/kg) and apomorphine 3 mg/kg (APO) on the swimming ability of BALB/c mice was tested in warm water (27±2° C.) on the third day following the treatment of MPTP. Swim-scores were recorded on a performance intensity scale of 0-3 for all the animals for 10 min. Results given are mean±0.05 or ^{#}p≦0.01 vs. control MPTP. Kruskal Wallis/Mann Whitney, n=6.

FIG. 20 shows the effect of different doses of Ptychopetalum olacoides extract (POEE) (25 and 50 mg/kg) and apomorphine 3 mg/kg (APO) on the swimming ability of BALB/c mice was tested in warm water (27±2° C.) on the seventh day following the treatment of MPTP. Swim-scores were recorded on a performance intensity scale of 0-3 for all the animals for 10 min. Results given are mean±S.E.M., *p≦0.05 or #p≦0.01 vs. control MPTP. Kruskal Wallis/Mann Whitney, n=6.

FIG. 21 shows the effect of different doses of Ptychopetalum olacoides extract (POEE) (25 and 50 mg/kg) and apomorphine 3 mg/kg (APO) on the swimming ability of BALB/c mice was tested in warm water (27±2° C.) on the fourteenth day following the treatment of MPTP. Swim-scores were recorded on a performance intensity scale of 0-3 for all the animals for 10 min. Results given are mean±S.E.M. Kruskal Wallis/Mann Whitney, n=6.

### DETAILED DESCRIPTION OF THE INVENTION

The invention will be described for the purposes of illustration only in connection with certain embodiments; however, it is to be understood that other objects and advantages of the present invention will be made apparent by the following description of the drawings according to the present invention. While a preferred embodiment is disclosed, this is not intended to be limiting. Rather, the general principles set forth herein are considered to be merely illustrative of the scope of the present invention and it is to be further understood that numerous changes may be made without straying from the scope of the present invention.

The present application describes extract of Ptychopetalum olacoides (olacaceae) as an active ingredient useful in preparing medicines, or pharmaceutical compositions, for the treatment or prevention of diseases, dysfunctions and disorders of the central nervous system, such as depressive and neurodegenerative disorders such as Alzheimer's and Parkinson's diseases.

The extract of this application not only fulfill several of the desired aspects described above for the ideal anticholinesterase agent, but also shows neuroprotective properties, consisting in a new prototypic anticholinesterase class of compounds. Adding to previous promnesic properties for the extract, it is noteworthy that the anticholinesterasic properties of these compounds are not the only pharmacodynamic basis for ameliorating diverse types of memories, since dopamine D₁, adrenergic β and serotonin 5HT_{2A} receptors are also involved in the identified promnesic and anti-amnesic properties. Moreover, the reversion of MK801-induced amnesia is suggestive of modulation of glutamate NMDA receptors, which play central roles in neuronal death and several neurodegenerative processes. Therefore the present extract, with promnesic, anti-amnesic and neuroprotective properties, containing innovative AChEI compounds that moreover modulate other receptors (glutamate, adrenergic, dopaminerfic and serotonergic) of renowned relevance for pro-cognitive, neuroprotective and antidepressive properties is perfectly in line with the cutting edge therapeutic approaches for neurodegenerative conditions. Neuroprotective properties of the compounds were demonstrated by a marked antioxidant activity in brain areas relevant to cognition, its capacity to protect hippocampus slices submitted to ischemia (oxygen and glucose deprivation model), the increased resilience to in vivo hypoxia, the reversion of tremors, akinesia and catalepsy induced by MPTP (an experimental model for Parkinson's Disease) and the reversal of β-amilóide changes (an experimental model for Alzheimer's Disease). The above mentioned modulation of neurotransmitters systems by the extract, especially glutamate and dopamine, are also relevant for neuroprotection.

As evidenced by the examples detailed below, the extract is efficacious in inhibiting brain cholinesterases, thereby augmenting the synaptic acetylcholine availability and consequently all functions dependent on cholinergic stimulation. Such functions include, neuroprotection, memory facilitation to various types of memory, reversal of amnesias induced by different neurotransmitter antagonists, protection against isquemic and oxygen reactive species.

The appropriate dosage of one or more active ingredients according to the present application can vary from about 0.001 mg/kg/day to about 5000 mg/kg/day, particularly from about 200 mg/kg/day to about 400 mg/kg/day, divided into one or more times a day.

Another embodiment of this application consists in a pharmaceutical composition containing an effective amount of extract of Ptychopetalum olacoides, in pharmaceutically acceptable excipients. The pharmaceutical compositions according to the present application can be liquid, semisolid or solid and can be adapted for any route of enteral or parenteral administration, either immediate release or modified. In particular achievement, said pharmaceutical composition is adapted for oral administration, particularly in the form of tablets, capsules, tinctures, emulsions, liposomes, microcapsules or nanoparticles.

Excipients suitable for the pharmaceutical composition of the present application are, for example and without limitation, those cited in the book Remington's Pharmaceutical Sciences, Mack Publishing publisher American, European Pharmacopoeia or the Brazilian Pharmacopoeia. Another object according to the present invention includes a method for preventing or treating disease, treating disease or neurodegenerative disorders such as Parkinson's disease and Alzheimer's disease or vascular dementia or cognitive deficits seen in older people, comprising supplying a patient in need with an effective amount of the extract o Ptychopetalum olacoides extract and/or a pharmaceutical composition containing such compounds.

The following examples serve to illustrate aspects of the present invention without having, however, any limiting character. We present tests with the extract of Ptychopetalum olacoides just for ease of presentation, without limitation only for this product.

### EXAMPLES

### I. Characterization of Acetylcholinesterase Inhibition in Relevant Brain Areas and Acetylcholinesterase Isoforms

### I.I. In Vitro

Because there is evidence that AChE-Is differentially inhibit the two major AChE molecular isoforms are found in the brain. (the cytosolic globular monomer (G1) and membrane bound globular tetramer (G4), and because these isomeric forms have different cellular distribution and functional significance in synaptic transmission (Brimijoin, S., 1983. Molecular forms of acetylcholinesterase in brain, nerve and muscle: nature, localization and dynamics. Prog Neurobiol 21:291-322), and since in healthy human brain, G1 and G4 AChE isoforms are responsible for 80% of total cholinesterase activity (Atack, J. R., Perry, E. K., Bonham, J. R., Candy, J. M., Perry, R. H., 1986. Molecular forms of acetylcholinesterase and butyrylcholinesterase in Alzheimer's disease resemble embryonic development: a study of molecular forms. Neurochem Int, 21:381-396), whereas in AD brain there is a selective loss of G4 and a relative sparing of G1 (Siek, G. C., Katz, L. S., Fishman, E. B., Korosi, T. S., Marquis, J. K., 1990. Molecular forms of acetylcholinesterase in subcortical areas of normal and Alzheimer disease brain. Biol Psychiatry 27, 573-580; Schegg, K. M., Harrington, L. S., Neilsen, S., Zweig, R. M., Peacock, J. H., 1992. Soluble and membrane-bound forms of brain acetylcholinesterase in Alzheimer's disease. Neurobiol Aging 13, 697-704), the following experiments characterize the inhibitory effect of Ptychopetalum olacoides extract in mouse hippocampus, frontal cortex, and striatum (brain areas relevant for cognition), taking into account specificities for G1 and G4 isoforms. Additionally, the nature of inhibition was determined in hippocampus.

AChE isoform sources: Male (CF1) adult (2 months old, 35-45 g) albino mice were sacrificed by guillotine, then the brains were quickly removed, cleaned with chilled saline, and cerebral structures dissected out over ice. The hippocampus, frontal cortex, and striatum were homogenized in 20, 10 and 20 volumes of buffer (0.01 M Tris-HCl buffer, pH-7.2 and 0.16 M sucrose), respectively, and centrifuged at 5000×g at 4° C. for 15 min (Eppendorf Centrifuge 5415R). The resulting supernatants were used as the G1 source (Das, A., Dikshit, M., Nath, C., 2001. Profile of acetylcholinesterase in brain areas of male and female rats of adult and old age. Life Sci 68, 1545-1555). The pellet was suspended in 1% Triton-X 100 (1% w/v in 0.5 M potassium phosphate buffer, pH-7.5) and centrifuged at 100,000×g at 4° C. in a Hitachi Refrigerated Centrifuge for 60 min. The supernatant was collected and used as the G4 source (Das, A., Dikshit, M., Nath, C., 2001. Profile of acetylcholinesterase in brain areas of male and female rats of adult and old age. Life Sci 68, 1545-1555).

AChE activity: Determination of AChE activity was adapted from the colorimetric method originally described by Ellman et al. (Ellman, G. L., Courtney, K. D., Andre, V. Jr., Featherstone, R. M., 1961. A new and rapid colorimetric determination of acetylcholinesterase activity. Biochem Pharmacol 7, 27-30). Briefly, 33 μL of 10 mM DTNB, 68 μL of Tris-HCl buffer, 100 μL of Ptychopetalum olacoides extract (0-1000 μg/mL), 33 μL of enzymatic material (3 μg/μL of protein for G1 or G4 AChE) were added to microplates followed by 33 μL of 0.8 mM ATC. The incubation solution contained the butyrylcholinesterase inhibitor tetraisopropyl pyrophosphoramide (iso-OMPA) at a final concentration of 100 μM in order to specifically measure AChE activity. The microplate was read at 415 nm every 30 s for 2.5 min (Microplate Reader Model 680, Bio-Rad Laboratories, UK). Experiments were performed in triplicate. AChE activities are expressed as μmol of acetylthiocholine hydrolyzed/hour/milligram of protein (μmol ATC/h/mg protein). The total enzymatic activity was determined and POEE IC₅₀ was obtained using the software package Prism Graph Pad 5.0 (Graph Pad Inc., San Diego, USA).

TABLE 1
Effect of Ptychopetalum olacoides extract on Km (μg/mL) and
Vₘₐₓ (μmol ATC/h/mg protein) of hippocampus G1 and G4 AChE.

| | | Vₘₐₓ | |
|---|---|---|---|
| | [POEE] | (μmol ATC/h/mg | Km |
| | (μg/mL) | protein) | (μg/mL) |
| G1 AChE | 0 | 5.86 | 7.91 |
| | 30 | 6.27 | 9.14 |
| | 100 | 6.11 | 10.63 |
| | 300 | 5.68 | 11.06 |
| | 1000 | 6.45 | 12.84 |
| G4 AChE | 0 | 6.05 | 8.29 |
| | 30 | 5.74 | 7.61 |
| | 100 | 4.11 | 6.0 |
| | 300 | 4.36 | 6.05 |
| | 1000 | 4.43 | 6.37 |

Vₘₐₓ and Km were measured on Lineweaver-Burk double reciprocal plots varying the concentration of the substrate ATC from 0.01 to 0.075 mM, and using increasing Ptychopetalum olacoides extractconcentrations (0, 30, 100, 300 and 1000 μg/mL) in hippocampus.

Kinetics analysis: To determine the type of enzyme inhibition, Lineweaver-Burk double reciprocal plots were produced by varying the concentration of the substrate ATC from 0.01 to 0.075 mM in the hippocampus. Plots were used to determine Km and Vₘₐₓ for Ptychopetalum olacoides extract using 0, 30, 100, 300 and 1000 μg/mL. Specific activities are expressed as μmol ATC/h/mg protein.

Protein assay: The protein content was determined as described by Lowry et al. (Lowry, O. H., Rosebrough, N. J., Farr, A. L., Randall, R. J., 1951. Protein measurement with the folin phenol reagent. J Biol Chem 193, 265-275), using bovine serum albumin (BSA) as standard.

Statistical analysis: The data were analyzed by one way analysis of variance (ANOVA) followed by Duncan's post hoc test. P<0.05 was adopted as the least significant level.

### Results:

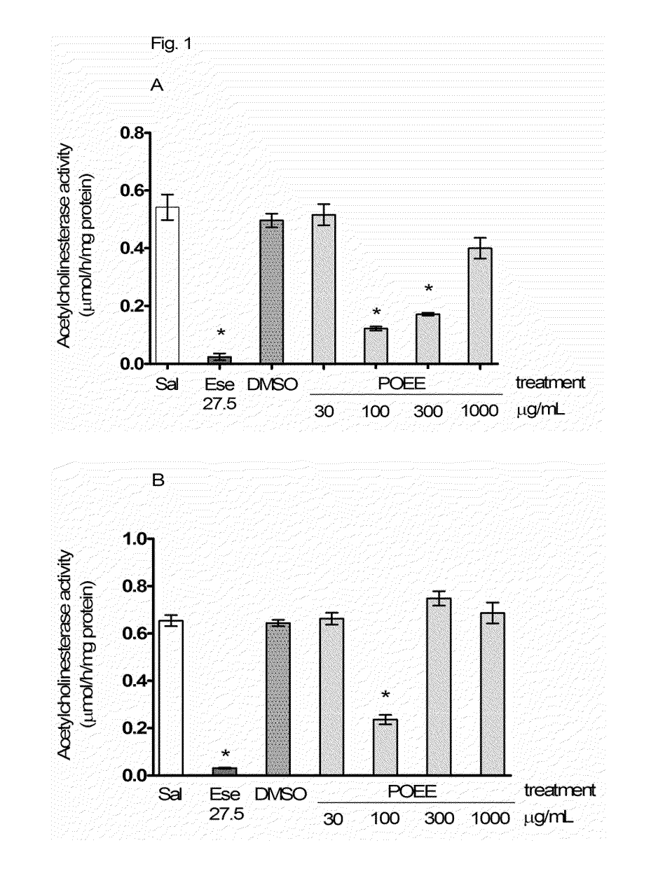

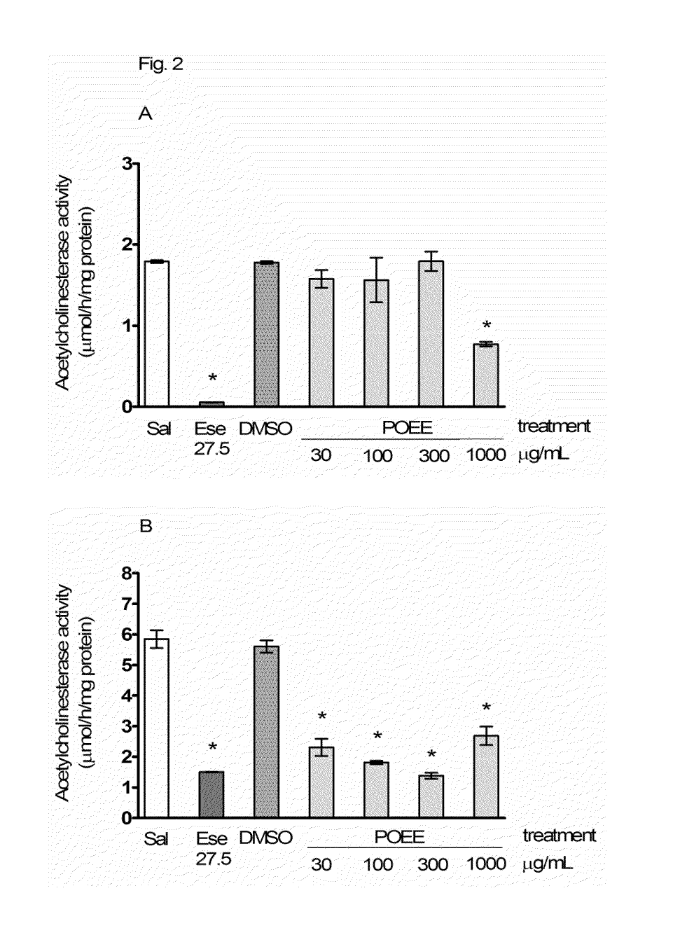

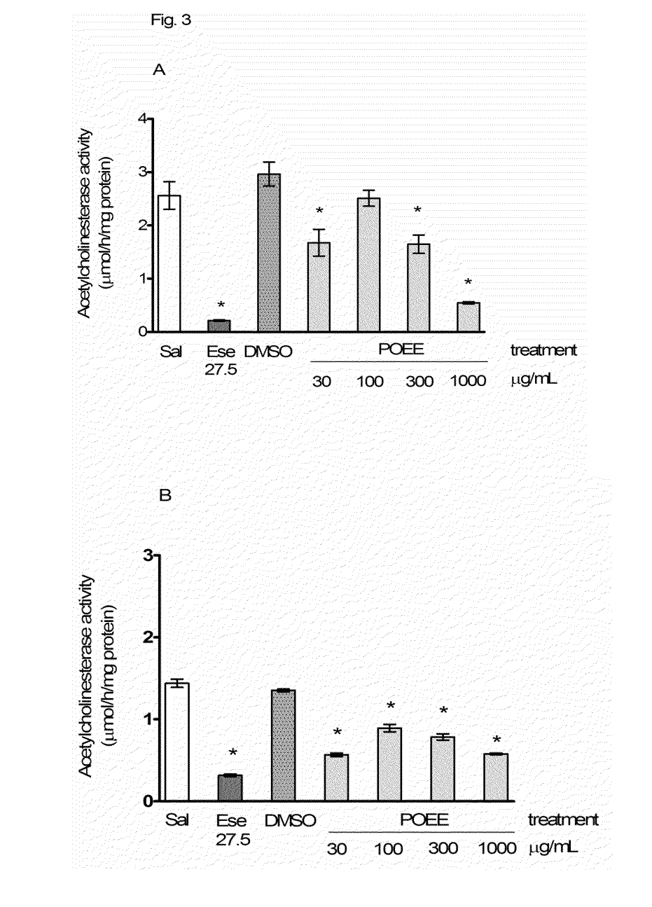

FIGS. 1, 2 and 3 show the effects of Ptychopetalum olacoides extract (0-1000 μg/mL) on G1 and G4 from hippocampus, frontal cortex and striatum, respectively. Ptychopetalum olacoides extract mostly inhibits (P<0.05) G1 in hippocampus (75%), and G4 in frontal cortex (58%) and striatum (75%).

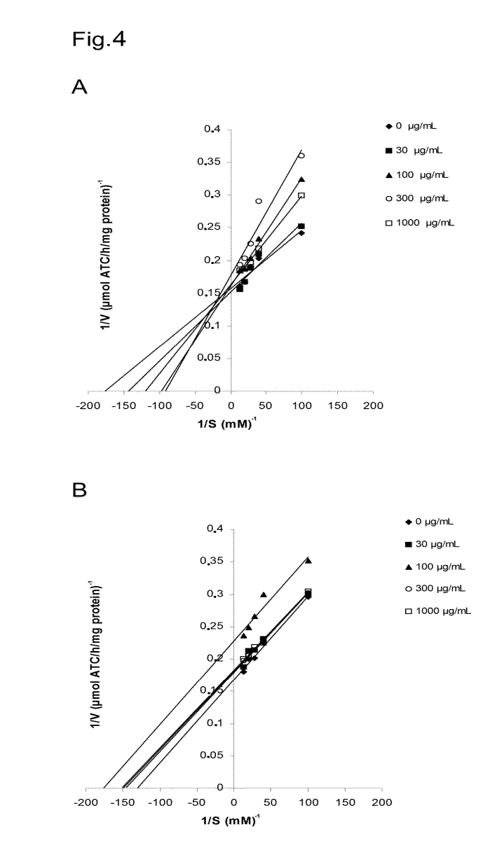

The kinetic analysis shown at Table 1 and FIG. 4 indicates that Ptychopetalum olacoides extract-induced inhibition in hippocampus is of a competitive nature for G1 and uncompetitive for G4.

### I.II. Ex-Vivo

A huge impediment for developing drugs for treating CNS diseases is the blood-brain barrier (BBB) and the extent to which a drug can readily penetrate the BBB determines its bioavailability (Anekonda, T. S. and Reddy, P. H., 2005. Can herbs provide a new generation of drugs for treating Alzheimer's disease? Brain Res Rev, 50: 361-376). The following experiments characterize histochemically the effect of different doses of Ptychopetalum olacoides extract on AChE activity at differents brain areas in mice orally treated with the compounds in Formula (I). The experiments prove the anticholinesterase effects of the compounds in Formula (I) in the desired sites after oral treatment.

Complementing the in vitro analysys, the effects of oral treatment with Ptychopetalum olacoides extract was analysed in the acetylcholinesterase isoforms G1 and G4 obtained from hippocampus, frontal cortex and striatum.

In addition, western blotting analysis were performed to measure the effects of Ptychopetalum olacoides extract in the acetylcholinesterase immunocontent in mice hippocampus and frontal cortex. The experiment show that the compounds do not affect the immunocontent, demonstrating that there is a functional inhibition rather than an effect in the enzyme syntheses.

Experimental groups and drug administration: Ptychopetalum olacoides extract was dissolved in a 20% DMSO solution. Groups of mice (N=5) were treated orally (by gavage) with a single dose of saline, galanthamine (5 mg/kg), DMSO 20%, and Ptychopetalum olacoides extract (300 or 800 mg/kg). All drugs were given as 0.1 mL/10 g body weight.

Preparation of Brain Slices: Ninety minutes after drug administration, under deep anesthesia (i.p. sodium thiopental 60 mg/kg), the animals were transcardially perfused with saline followed by a cold 4% paraformaldehyde solution in 0.1 M phosphate buffer (PB), pH 7.4. After complete perfusion brains were removed, post-fixed in the same fixative solution at room temperature for 4 hours, and sectioned (coronal sections; 50 μm) with a vibratome (Leica, Germany). The sections were collected in PB.

Histochemistry procedure: The free-floating sections were carefully washed in 0.1 M tris maleato buffer, pH 6 (TMB) and processed for AChE histochemistry as described by Karnovsky and Roots (Karnovsky, M. J. and Roots, L., 1964. A “direct-coloring” thiocoline method for cholinesterases. J Histochem Cytochem, 12: 219-221). Each section was incubated during 4 h at room temperature and protected from light in microplates filled with 3 ml of the following solution: acetylthicholine iodide 2.5 mM, TMB 0.1 M sodium citrate, 30 mM copper sulfate, 5 mM potassium ferricyanide in distilled water. Cupric ferrocyanide (Karnovsky's precipitate) and cuprous thiocholine iodide (resulting from ferricyanide and cupric ions reduced by thicholine) are the expected histochemical products. Immediately after incubation, sections were rinsed 3 times in TMB, dehydrated in ethanol, cleared with xylene, and covered with balsam and a coverslip. Experiments included brains from all experimental groups, and the entire procedure were carefully executed to ensure that all sections were submitted to exactly the same histological steps, identical incubation medium and same incubation time. Therefore eventual differences in histochemistry reaction or changes in the background levels among the various groups were kept as minute as possible.

Optical densitometry: Hippocampus (CA1 and CA3), striatum (caudate putamen, CPu), basolateral amygdaloid nucleus anterior (BLA) and lateral entorhinal cortex (LEnt) were identified according to Franklin and Paxinos Atlas (Franklin, K. B. J. and Paxinos, G. T., 1996. The mouse brain in stereotaxic coordinates, Academic Press, San Diego), with the following coordinates: interaural 2.34 at 1.10 mm, bregma −1.46 at −2.70 mm for CA1/CA3, interaural 2.34 at 1.50 mm, bregma −1.46 at −2.30 mm for CPu, BLA and LEnt. These areas were selected for its relevance to cognition and/or abundant cholinergic afference. The intensity of the AChE histochemistry was assessed by semi-quantitatively denstitometric analysis (Xavier, L. L., Viola, G. G., Ferraz, A. C., Da Cunha, C., Deonizio, J. M., Netto, C. A., Achaval, M., 2005. A simple and fast densitometric method for the analysis of tyrosine hydroxylase immunoreactivity in the substantia nigra pars compacta and in the ventral tegmental area. Brain Res Brain Res Protoc, 16:58-64; Winkelmann-Duarte, E. C., Todeschin, A. S., Fernandes, M. C., Bittencourt, L. C., Pereira, G. A., Samios, V. N., Schuh, A. F., Achaval, M. E., Xavier, L. L., Sanvitto, G. L., Mandarim-de-Lacerda, C. A., Lucion, A. B., 2007. Plastic changes induced by neonatal handling in the hypothalamus of female rats. Brain Res, 19:20-30), using a Nikon Eclipse E-600 (Japan, Tokyo) microscope coupled to a Pro-Series High Performance CCD camera and the Image Pro Plus Software 6.0 (Media Cybernetics, CA, USA). The digitized images from selected areas (left and right brain sides) were converted to an 8-bit gray scale (0-255 gray levels), and lighting conditions and magnifications were held constant throughout the analysys. 100× magnification was used for CA1 and CA3 and 40× magnification for CPu, BLA and LEnt. The optical density (OD, pixels) was measured in 325.5 μm2 squares delimited at CA1 and CA3, and 8053.9 μm2 squares at CPu, BLA and LEnt. Selected squares were free from blood vessels or procedure-induced tissue marks. ODs were obtained from at least 40 slices from each animal, with the average OD/area used as individual OD. Investigators were unaware of the slice source (experimental groups) being analysed. The optical density (OD) was calculated in accordance with our previous published protocol (Xavier, L. L., Viola, G. G., Ferraz, A. C., Da Cunha, C., Deonizio, J. M., Netto, C. A., Achaval, M., 2005. A simple and fast densitometric method for the analysis of tyrosine hydroxylase immunoreactivity in the substantia nigra pars compacta and in the ventral tegmental area. Brain Res Brain Res Protoc, 16:58-64).

Preparation of total and synaptosomal membranes: Ninety minutes after drug administration mice were sacrificed by decapitation and the hippocampus and frontal cortex were dissected out in ice to obtain total and percoll purified synaptosomal membranes as previously described (Cunha, R. A., Johansson, B., Constantino, M. D., Sebastião, A. M., Fredholm, B. B., 1996. Evidence for high-affinity binding sites for the adenosine A2A receptor agonist [3H]CGS 21680 in the rat hippocampus and cerebral cortex that are different from striatal A2A receptors. Naunyn Schmiedeberg's Arch Pharmacol 353, 261-271). Briefly, brain structures were dissected and homogenized (5%, w/v) in 0.32 M sucrose, 10 mM HEPES, pH 7.4 (sucrose buffer), using a homogenizer. The suspension was centrifuged at 3,000 rpm for 2 min, and supernatants were spun at 14,000 rpm for 12 min. The upper white layer of the pellet (P2) was removed and resuspended in 5% SDS with a protease cocktail inhibitor (Sigma, São Paulo/Brazil). Alternatively, a purified hippocampal synaptosomal suspension was isolated using the Percoll method described elsewhere (Dunkley et al., 1986) by resuspending P2 in 500 μL of 45% (v/v) Percoll solution in Krebs (140 mM NaCl, 5 mM KCl, 25 mM HEPES, 1 mM EDTA, 10 mM glucose, pH 7.4), centrifuged at 14,000 g for 20 minutes min at 4° C. The top layer (synaptosomal fraction) was collected in 1 mL Krebs solution, washed and the synaptosomal fraction was centrifuged again at 14,000×g for 2 min at 4° C. and the pellet was ressuspended in 5% SDS with a protease cocktail inhibitor (Sigma, São Paulo/Brazil). The samples were frozen at −70° C.

Western blotting analysis: After defrost, the protein determination of the synaptosomal and total membranes from hippocampus and frontal cortex were carried out by using Bicinchoninic acid assay using bovine serum albumin (BSA) as standard (Pierce, São Paulo/Brazil). Samples were diluted to a final protein concentration of 2 μg/μL in SDS-PAGE buffer; 40 μg (20 μL) of samples and 20 μL of a dual color pre-stained molecular weight standard (Bio-Rad, Porto Alegre, Brazil) were separated by SDS-PAGE (10% concentrating gel). After electro-transfer, the membranes were blocked with Tris-buffered saline 0.1% Tween-20 (TBS-T) containing 3% BSA. After blocking, the membranes were incubated for 24 h at 4° C. with mouse anti-AChE antibody (1:1000, Chemicon Int., São Paulo/SP, Brazil). After primary antibody incubation, membranes were washed in TBS-T and incubated with horseradish peroxidase-conjugated secondary antibodies for 2 h at room temperature and developed with ECL (Amersham, São Paulo/Brazil). The autoradiographic films were scanned and densitometric analyses were performed using public domain NIH Image Program (http://rsb.info.nih.gov/nih-image/). As an additional control of the protein loading, membranes were stained with Ponceau S or mouse anti-GAPDH antibody (1:1000).

AChE activity: Determination of AChE activity was adapted from the colorimetric method originally described by Ellman et al. (Ellman, G. L., Courtney, K. D., Andre, V. Jr., Featherstone, R. M., 1961. A new and rapid colorimetric determination of acetylcholinesterase activity. Biochem Pharmacol 7, 27-30) as described above for in vitro studies.

Statistics: The data are expressed as means±S.E.M. One way analysis of variance (ANOVA) followed by the Duncan multiple group comparison was used to image analysis. Paired Student t-test was used to validate methods against positive controls. Significance was set at P<0.05.

### Results:

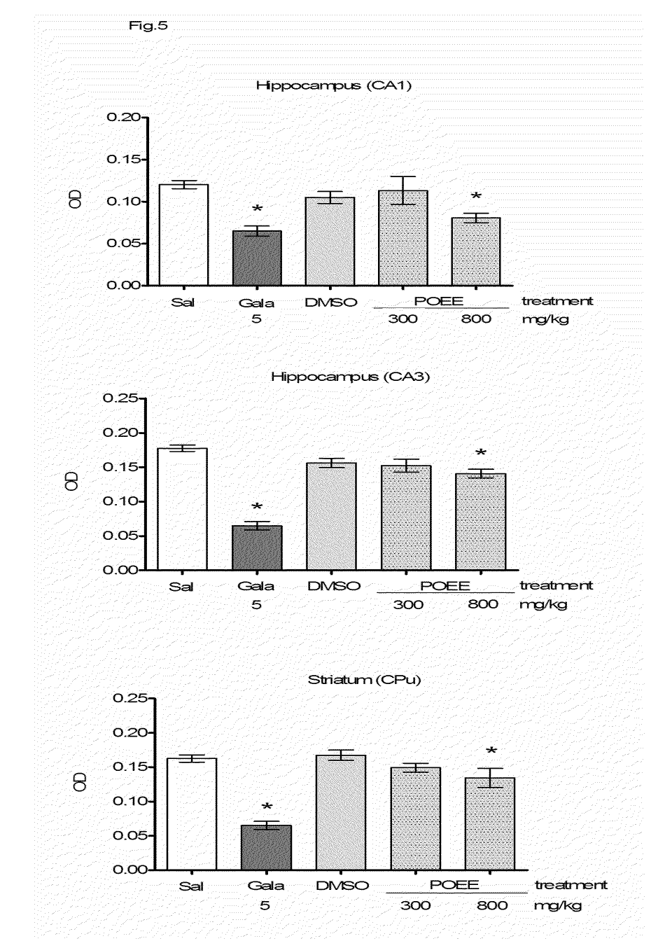

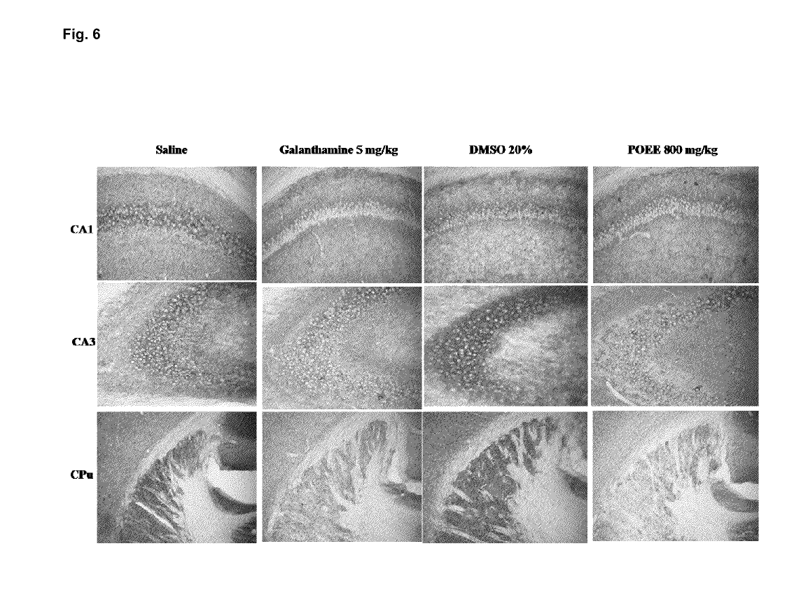

FIG. 5 shows AChE histochemistry intensity, expressed in optical density (OD). Ptychopetalum olacoides extract 800 mg/kg significantly (P<0.05) decreased OD in CA1 (0.08±0.01), CA3 (0.14±0.01), and CPu (0.13±0.01), as compared to DMSO (CA1: 0.10±0.01; CA3: 0.17±0.01; and CPu: 0.17±0.01). AChE inhibition corresponded to 33%, 20% and 17% on CA1, CA3 and CPu, respectively. FIG. 6 illustrated this result.

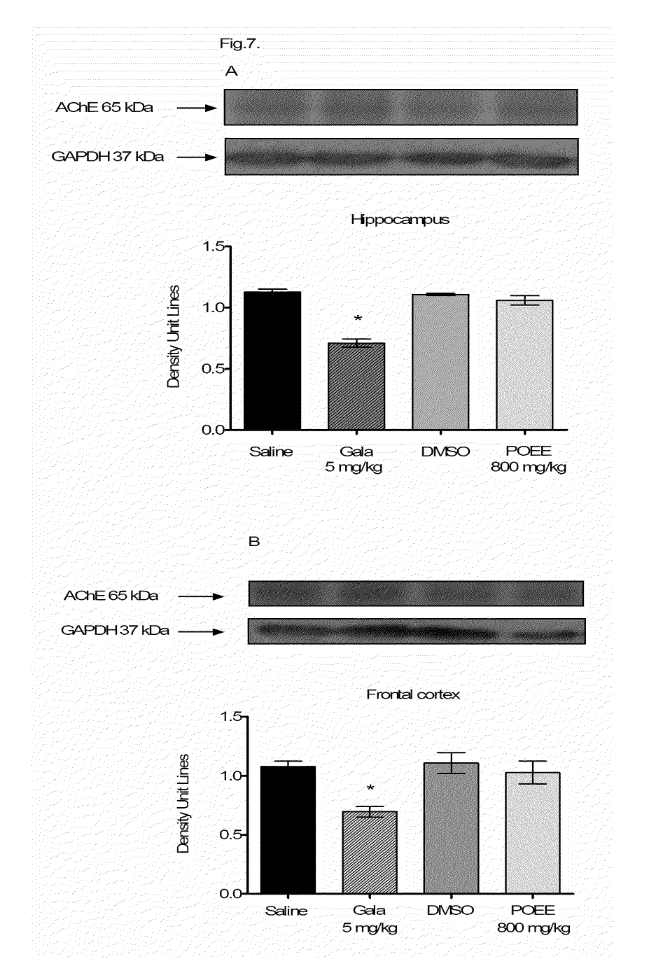

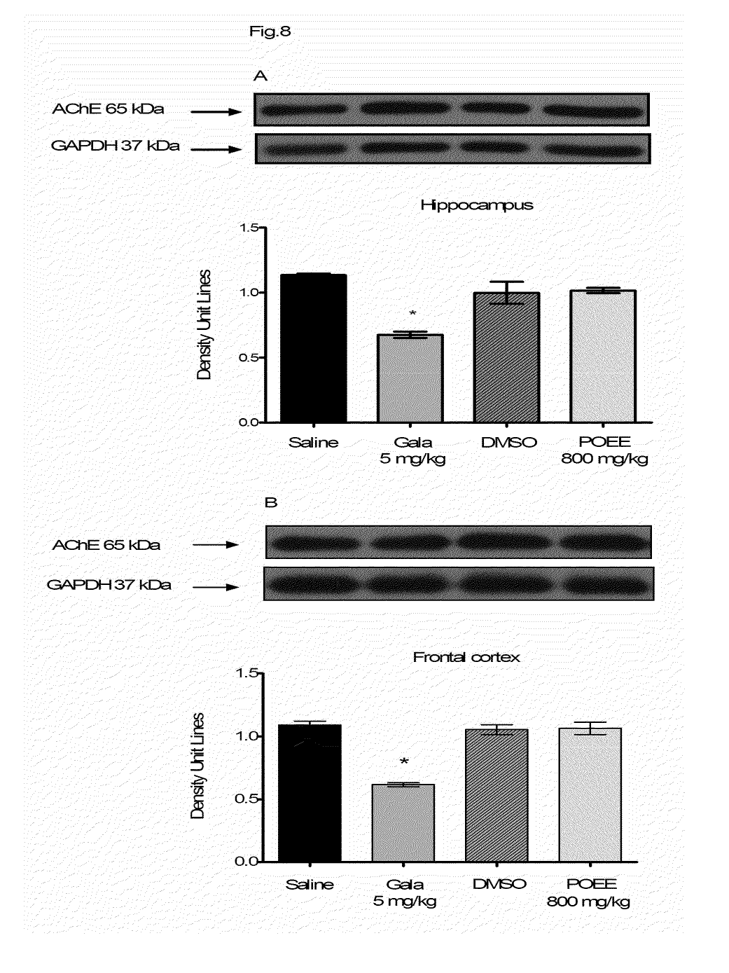

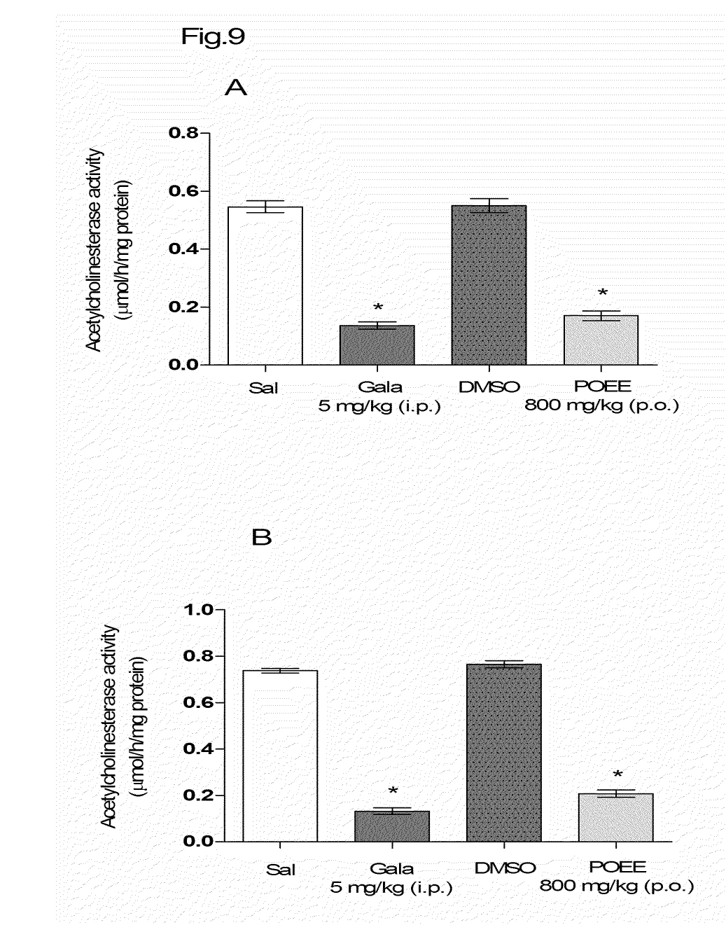

FIGS. 7A-B, 8A-B and 9A-B show the effects of Ptychopetalum olacoides extract (800 mg/kg) on G1 and G4 from hippocampus, frontal cortex and striatum, respectively. Ptychopetalum olacoides extract mostly inhibits (P<0.05) G1 and G4 (−70%) in hippocampus, and G4 in frontal cortex (62%) and striatum (75%).

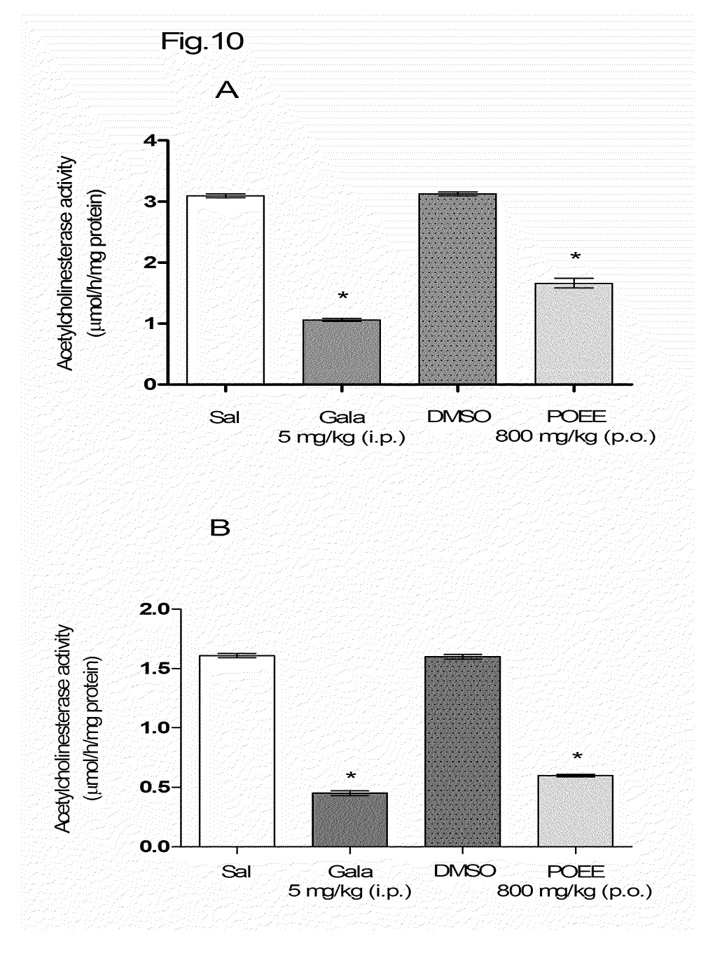

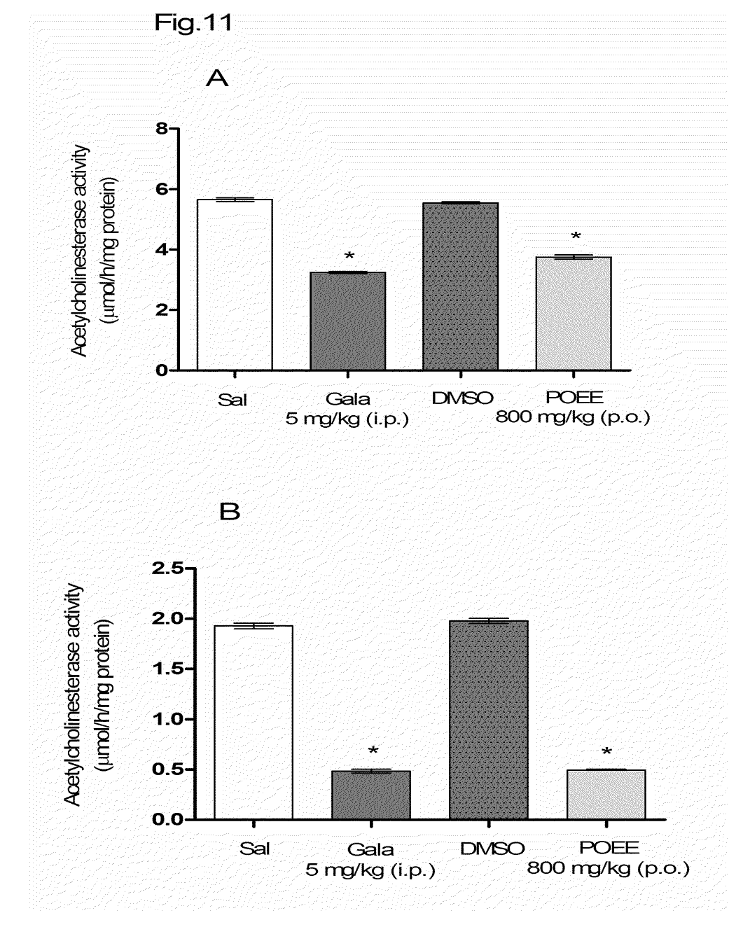

FIGS. 10A-B and 11A-B show the effects of Ptychopetalum olacoides extract on the AChE immunocontent in total membranes and synaptosomal membranes from the hippocampus and frontal cortex. No significant changes were induced by Ptychopetalum olacoides extract 800 (mg/kg) in hippocampus and frontal cortex total membranes or synaptosomal membranes.

### II. Alzheimer's Disease Model

Alzheimer's disease is pathologically characterized by the presence of extracellular plaques of β-amyloid peptide (Aβ) (Glenner, G. G. and Wong, C. W., 1984. Alzheimer's disease: initial report of the purification and characterization of a novel cerebrovascular amyloid protein. Biochem Biophys Res Commun, 120(3):885-90; Masters, C. L., Multhaup, G., Simms, G., Pottgiesser, J., Martins, R. N., Beyreuther, K., 1985. Neuronal origin of a cerebral amyloid: neurofibrillary tangles of Alzheimer's disease contain the same protein as the amyloid of plaque cores and blood vessels. EMBO J. 4(11): 2757-63), and intracellular tangles of hyperphosphorylation tau protein (Ballatore, C., Lee, V. M., Trojanowski, J. Q., 2007. Tau-mediated neurodegeneration in Alzheimer's disease and related disorders. Nat Rev Neurosci, 8(9): 663-72; Braak, H. and Braak, E., 1998. Evolution of neuronal changes in the course of Alzheimer's disease. J Neural Transm Suppl, 53:127-40). These changes result in loss of forebrain cholinergic neurons and pronounced acetylcholine reduction (Bartus, R. T., Dean, R. L., Beer, B., Lippa, A. S., 1982a. The cholinergic hypothesis of geriatric memory dysfunction. Science 217, 408-417), although, the connections between these pathological hallmarks and mechanism by which Aβ causes neuronal injury and cognitive impairment is not yet clearly understood (Pimplikar, S. W., 2009. Reassessing the amyloid cascade hypothesis of Alzheimer's disease. Int J Biochem Cell Biol, 41(6): 1261-8; Jhoo, J. H., Kim, H. C, Nabeshima, T., Yamada, K., Shin, E. J., Jhoo, W. K., Kim, W., Kang, K. S., Jo, S. A., Woo, J. I., 2004. Beta-amyloid (1-42)-induced learning and memory deficits in mice: involvement of oxidative burdens in the hippocampus and cerebral cortex. Behav Brain Res, 155(2):185-96).

The following experiments investigate whether chronic oral administration of Ptychopetalum olacoides extract protects mice from the learning and memory deficits induced by intracerebroventricular (icv) β-amyloid protein-(1-42), a mice model of AD. Experiments show that treatment with Ptychopetalum olacoides extract for 15 days already attenuates the consequences of icv β-amyloid.

BDNF (brain-derived neurotrophic factor) and its receptor are involved in cholinergic cell survival, maintenance and axonal growth (Bibel, M. and Barde, Y. A., 2000. Neurotrophins: key regulators of cell fate and cell shape in the vertebrate nervous system. Genes Dev, 14(23): 2919-37; Chao, M. V., 2003. Neurotrophins and their receptors: a convergence point for many signaling pathways. Nat Rev Neurosci 4: 299-309), stimulate choline acetyltransterase (ChAT) activity (Auld, D. S., Mennicken, F., Quirion, R., 2001. Nerve growth factor rapidly induces prolonged acetylcholine release from cultured basal forebrain neurons: differentiation between neuromodulatory and neurotrophic influences. J Neurosci 21: 3375-3382; Berse, B., Szczecinska, W., Lopez-Coviella, I., Madziar, B., Zemelko, V., Kaminski, R., Kozar, K., Lips, K. S., Pfeil, U., Blusztajn, J. K., 2005. Expression of high affinity choline transporter during mouse development in vivo and its upregulation by NGF and BMP-4 in vitro. Brain Res Dev Brain Res 157: 132-140) and have been implicated in neurodegenerative disorders (Mattson, M. P. and Magnus, T., 2006. Ageing and neuronal vulnerability. Nat Rev Neurosci 7: 278-294). Therefore, an additional experiment was performed to evaluate whether chronic oral administration of Ptychopetalum olacoides extract alters BDNF levels in hippocampus. The experiment show that BDNF is not altered either by β-amyloid in this mice model of AD, nor by Ptychopetalum olacoides extract treatment for 15 days. Therefore the protection afforded by the Ptychopetalum olacoides extract treatment against β-amyloid induced cognitive deficits is more likely to be mediated by its anticholinesterase properties and the same receptors (D1 and β that mediate its promnesic activity.

Experimental design: Ptychopetalum olacoides extract was dissolved in a DMSO 20% (v/v) solution. After Aβ₁₋₄₂ or PBS administration i.c.v, groups of mice (N=12) were treated orally (by gavage) for 14 consecutives days with a single dose of saline (0.9 g %), DMSO 20%, or Ptychopetalum olacoides extract (800 mg/kg). All drugs were given as 0.1 mL/10 g body weight. Cognitive deficit was assessed using step-down inhibitory avoidance task and hippocampal BDNF levels was measured by immunoassay. The effects of treatments on locomotion were evaluated.

Intracerebroventricular injection of β-Amyloid peptide: The administration of β-amyloid (1-42) peptide fragment (Aβ₁₋₄₂) was performed according to the procedure established by Laursen & Belknap (Laursen, S. E. and Belknap, J. K., 1986. Intracerebroventricular injections in mice. Some methodological refinements. J Pharmacol Methods, 16(4): 355-7). The peptide was prepared as stock solution at a concentration 500 μM in sterile 0.1M phosphate-buffered saline (PBS) (pH 7.4), aliquot and stored at −20° C. Aβ₁₋₄₂ (400 μmol/mouse) or control solution (PBS) were administered by intracerebroventricular (i.c.v.) route using a microsyringe with a 28-gauge stainless-steel needle 3.0 mm long (Hamilton). In brief, the needle was inserted unilaterally 1 mm to the right of the midline point equidistant from each eye, at an equal distance between the eyes and the ears and perpendicular to the plane of the skull. The injection volume (4 μL) of Aβ₁₋₄₂ or PBS was delivered gradually. Mice exhibited normal behaviour within 1 min after injection. The injection placement or needle track was visible and was verified at the time of dissection. The present Aβ₄₁₋₄₂ is comparable to that of previous literature (Kim, H. S., Cho, J. Y., Kim, D. H., Yan, J. J., Lee, H. K., Suh, H. W., Song, D. K., 2004. Inhibitory Effects of Long-Term Administration of Ferulic Acid on Microglial Activation Induced by Intracerebroventricular Injection of β-Amyloid Peptide (1-42) in Mice. Biol Pharm Bull, 27(1): 120-121; and Yan, J. J., Cho, J. Y., Kim, H. S., Kim, K. L., Jung, J. S., Huh, S. O., Suh, H. W., Kim, Y. H., Song, D. K., 2001. Protection against b-amyloid peptide toxicity in vivo with long-term administration of ferulic acid. British J Pharmacol 133: 89-96). The behavioral performance was evaluated 14 days after the only administration of Aβ.

Locomotion: Twenty four hours before step-down inhibitory avoidance task, the locomotor activity was avaliated. Number of crossings were automatically recorded in activity cages (45×25×20 cm, Albarsch Electronic Equipment), equipped with three parallel photocells (Creese, I., Burt, D. R., Snyder, S. H., 1976. Dopamine receptor binding predicts clinical and pharmacological potencies of antischizophrenic drugs. Science, 192(4238):481-483). Mice were individually placed in the activity cages and the crossings were recorded for 8 min, being the first 3 min of exploratory and the 5 final minutes of locomotor activity.

Step-down inhibitory avoidance performance: The test used was adapted from Netto and Izquierdo (Netto, C. A. and Izquierdo, I., 1985. On how passive is inhibitory avoidance. Behav Neural Biol 43: 327-330) and from Maurice et al. (Maurice, T., Hiramatsu, M., Itoh, J., Kameyama, T., Hasegawa, T., Nabeshima, T., 1994. Behaviour evidence for modulation role of σ ligands in memory process. I. Attenuation of dizocilpine (MK-801)-induced amnesia. Brain Res 647: 44-56). Fourteen days after Aβ₁₋₄₂ injection, mice were trained on a one-trial step down inhibitory avoidance task. Mice were habituated in the dim lighted room for at least 60 min before the experiments. The inhibitory avoidance apparatus was a plastic box (30 cm×30 cm×40 cm), with a platform (5 cm×5 cm×4 cm) fixed in the center of the grid floor. Each mouse was placed on the platform and the latency to step down (four paws on the grid), was automatically recorded in the training and test sessions. In the training session, mice received a scrambled foot shock (0.3 mA for 15 s) upon stepping down. The test session was performed 24 h later (long-term memory—LTM), with the same procedure except that no shock was administered after stepping down; an upper cut-off time of 300 s was set.

Analysis of BDNF tissue levels: To measure the amount of BDNF in each sample, Promega BDNF Emax ImmunoAssay System was employed (Promega Co., Madison, Wis., USA), according to manufacturer's recommendations. Briefly, hippocampus (n=5 per group) were individually homogenized in lysis buffer [containing, in mM: 137 NaCl, 20 Tris-HCl (pH 8.0), Igepal (1%), glycerol (10%), 1 PMSF, 0.5 sodium vanadate, 0.1 EDTA and 0.1 EGTA] and centrifuged at 14,000 rpm at 4° C. during 3 min. Supernatant was diluted in sample buffer and incubated on 96-well flat-bottom plates previously coated with anti-BDNF monoclonal antibody (1:1000). After blocking (with Promega 1× Block and sample buffer), plates were incubated with polyclonal anti-human antibody for 2 h and horseradish peroxidase for 1 h. Then, color reaction with tetramethyl benzidine was quantified in a plate reader at 450 nm; the standard BDNF curve ranged from 0-500 pg/mL.

Protein assay: Total protein concentration was measured by Lowry's method using bovine serum albumin as a standard (Lowry, O. H., Rosebrough, N. J., Farr, A. L., Randall, R. J., 1951. Protein measurement with the folin phenol reagent. The Journal of Biological Chemistry 193, 265-275).

Statistical analysis: Locomotor activity and BDNF levels are expressed as mean±SEM and statistical significance were determined by one-way ANOVA followed by post hoc Duncan's test. Step-down latencies are expressed as medians [interquartile ranges]. Data were analyzed by Kruskal-Wallis non-parametric analysis of variance; comparisons among treatment groups were completed through Mann-Whitney U-test (two-tailed), and within treatment groups by the Wilcoxon test. P<0.05 was considered statistically significant.

### Results:

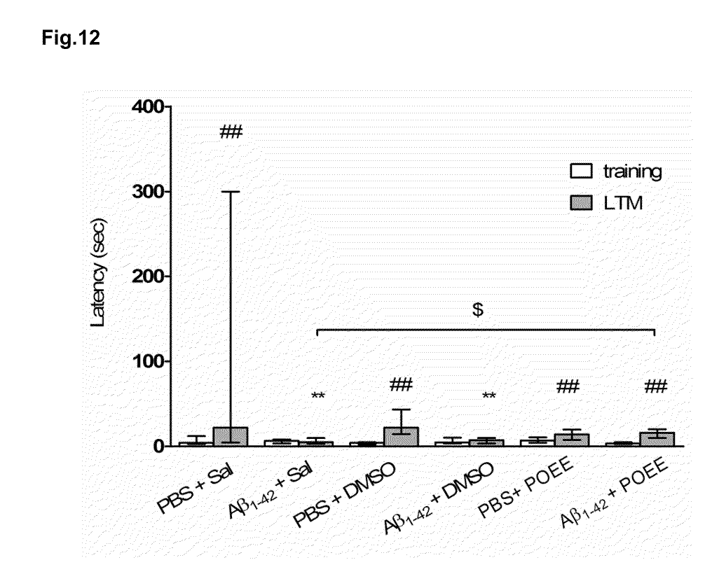

FIG. 12 shows that icv Aβ₁₋₄₂ (400 μmol/mouse) impaired mice performance in the inhibitory avoidance (P<0.05), and that Ptychopetalum olacoides extract treatment for 15 days attenuated such impairment (P<0.05).

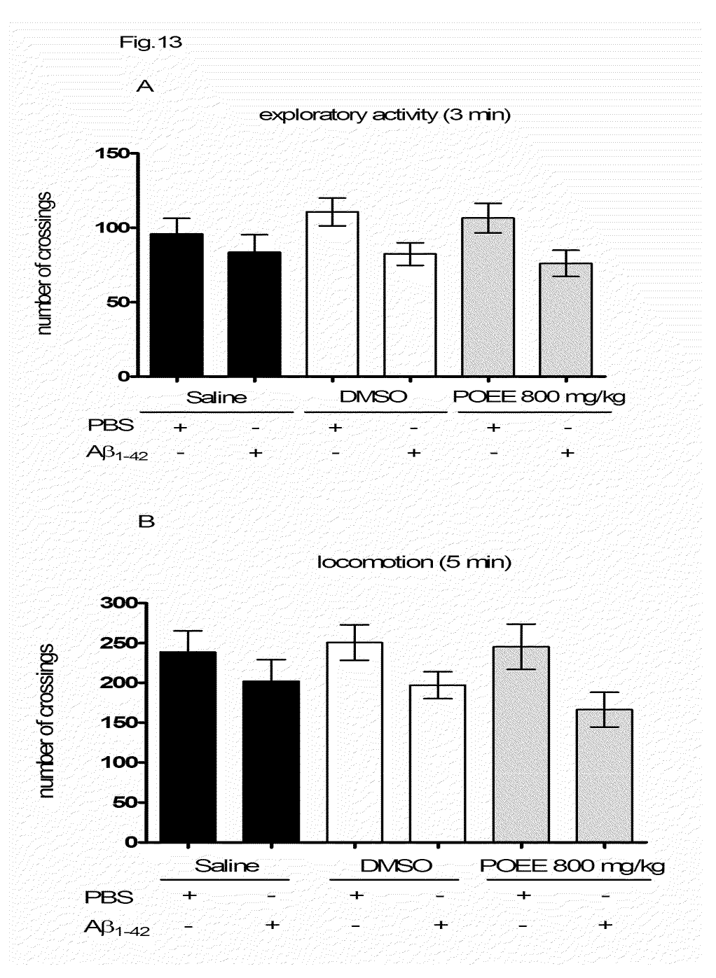

FIG. 13 shows that Ptychopetalum olacoides extract did not alter the locomotion, which could mask the results of such analysis.

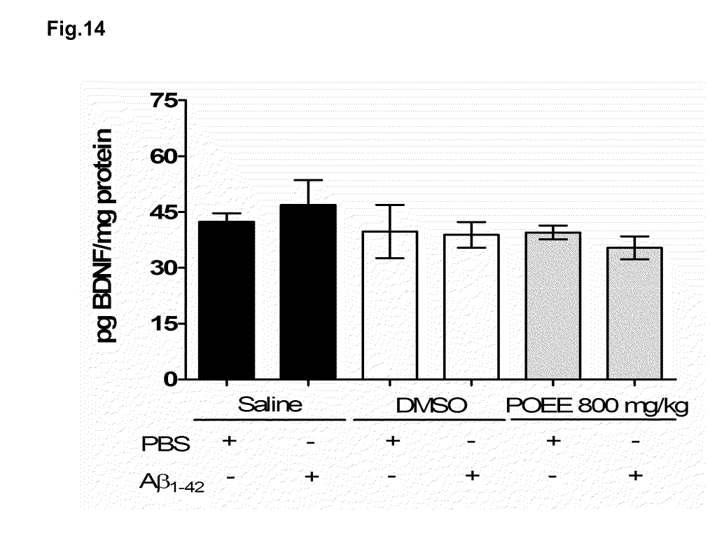

FIG. 14 shows that no significant changes were seen in hippocampus BDNF levels either with Aβ₁₋₄₂ or Ptychopetalum olacoides extract.

### III. Parkinson's Disease Model

Parkinson's Disease (PD) is characterized by a progressive and irreversible loss of dopamine neurons at the nigro-striatal area. The reasons for this specific death are unclear, it has been suggested that neuronal death is linked to excitotoxic lesions, oxidative stress and a byproduct of dopamine metabolism (Martignoni, E., Blandini, F., Godi, L., Desideri, S., Pacchetti, C., Mancini, F., Nappi, G., 1999. Peripheral markers of oxidative stress in Parkinson's Disease. The role of L-Dopa. Free Radic Biol Med., 27(3-4):428-37; Dauer, W., Przedborski, S., 2003. Parkinson's disease: mechanisms and models. Neuron, 39(6):889-909).

The MPTP (1-methyl-4-fenyl-1,2,3,6-tetrahydropiridine) neurotoxin mimics in animals de effects of PD, reproducing various symptoms (such as akinesica, rigidity and catalepsy) as well as the neurodegeneration at substantia nigra (Beal, M. F., 2001. Experimental models of Parkinson's disease. Nat Rev Neurosci 2:325-34), consolidating a valid animal model of PD.

Given that traditional uses of P. olacoides include tremors, and considering the antioxidative and neuroprotective properties of the extract from which the compounds in formula (I) were obtained, the purpose of the following experiments was to evaluate the effects of such compounds in the MPTP model of PD in mice.

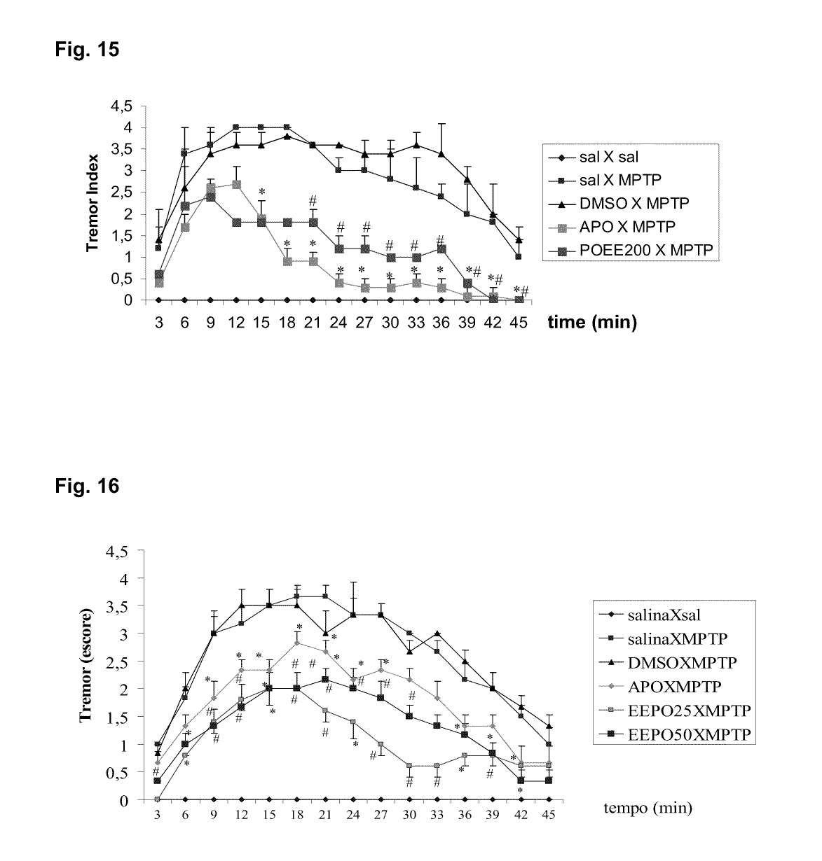

FIG. 15 shows that acute treatment with Ptychopetalum olacoides extract reduced the intensity of tremors at 21 min (P≦0.01), as well as its duration (from 45 min in controls to 39 min for C57BL/6 mice treated with the compounds).

FIG. 16 shows that acute treatment with Ptychopetalum olacoides extract reduced the intensity of tremors; while control Balb/c mice treated with MPTP show a continuous tremor of 3.7±0.0 at 18 min, and tremors that lasted over 45 min (H(5)=27.8, P<0.01). Likewise with C57BL/6 mice, Balb/c mice treated with Ptychopetalum olacoides extract 25 mg/kg showed tremors with median score of 1.4±0.2 at 9 min (P≦0.01). Total tremor duration was not affected by Ptychopetalum olacoides extract or apomorphine.

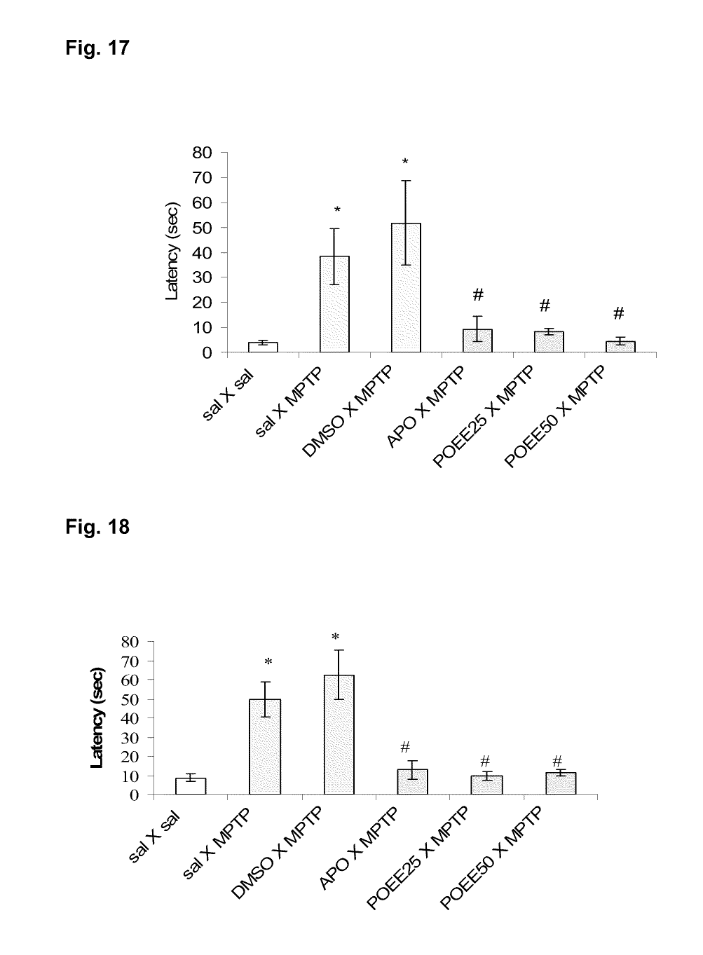

FIG. 17 shows that acute treatment with Ptychopetalum olacoides extract reduced akinesia (H(5)=23.5, P<0.01), with latency equal to 9.9±2.1 seg in mice treated with 25 mg/kg kg, and 11.1±1.7 seg in those treated with 50 mg/kg, in comparison to 62.5±12.8 of controls.

FIG. 18 shows that acute treatment with Ptychopetalum olacoides extract reduced catalepsy (H(5)=26.1, P<0.01) with 8.2±1.2 seg in mice treated with 25 mg/kg, and 4.5±1.6 seg in those treated with 50 mg/kg, in comparison to 51.8±16.7 of controls.

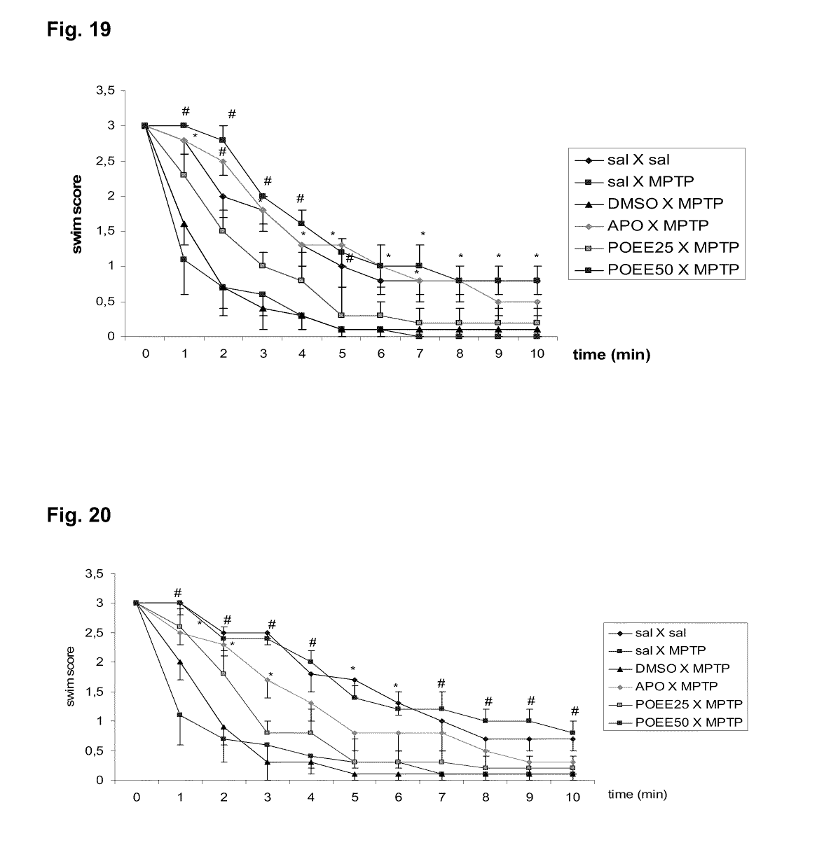

The detrimental effects of MPTP in Balb/C mice swimming capacity can be seen in FIGS. 19-21, at the 3^{rd} day (H(5)=20.7, P<0.01), and 7^{th} day (H(5)=19.6, P<0.01) post treatment, with complete recovery after 14 days.

FIG. 19 shows that acute treatment with Ptychopetalum olacoides extract 50 mg/kg protected mice from the MPTP effect at day 3 post MPTP.

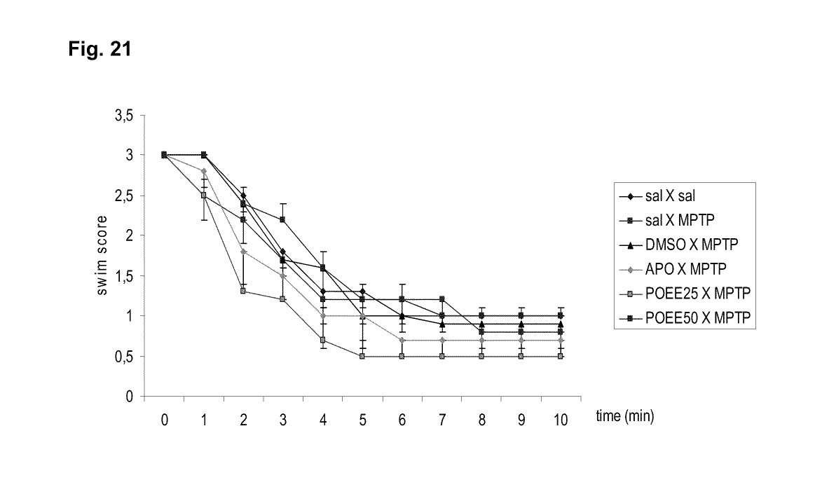

FIG. 20 shows that acute treatment with Ptychopetalum olacoides extract 50 mg/kg protected mice from the MPTP effect at day 7 post MPTP.

FIG. 21 shows that at day 14 post MPTP there are no significant differences in treatment groups.

Animals: Male adult mice, C57BL/6 and BALB/c strain (FEEPS) were used and maintain with water and foot ad libitum under controlled environment.

Treatments in C57BL/6: MPTP (1-methyl-4-phenyl-1,2,3,6-tetrahydropyridine) 50 mg/kg (2×25 mg/kg) was administered intraperitonially (i.p.), twice, 1 h apart. Saline, DMSO 20%, Ptychopetalum olacoides extract 200 mg/kg (2×100 mg/kg) and apomorphine 3 mg/kg (2×1.5 mg/kg) were given 30 min before each MPTP administration. The animals received the first injections at 8:00 h and the second at 9:00 h. The volume of injection was 0.1 ml/g body weight. N=5.

Treatments in BALB/c: MPTP (1-methyl-4-phenyl-1,2,3,6-tetrahydropyridine) 60 mg/kg (2×30 mg/kg) was administered intraperitonially (i.p.), twice, 16 h apart. Saline, DMSO 20%, Ptychopetalum olacoides extract 50 mg/kg (2×25 mg/kg), POEE 25 mg/kg (2×12.5 mg/kg) and apomorphine 3 mg/kg (2×1.5 mg/kg) were given 30 min before each MPTP administration. The animals received the first injections at 17:00 h and the second at 09:00 h the next day. The volume of injection was 0.1 ml/g body weight. N=6.

Tremor in C57BL/6 and in BALB/c: Tremors were observed immediately after the administration of the second MPTP dose, with animals placed in a clear Plexiglas box (20 cm×20 cm×20 cm) for 45 min; tremor scores were noted every 3 min, with the highest score considered for the period. Tremors were quantified on a modified intensity-score basis in a scale of 0-5 as described earlier (Hoabam, R., Sindhu, K. M., Chandra, G., Mohanakumar, K. P., 2005. Swim-test as a function of motor impairment in MPTP model of Parkinson's disease: a comparative study in two mouse strains. Behavioural Brain Research 163:159-167): 0, no tremor; 1, occasional muscle twitches or slight tremor which is barely visible at the head region; 2, moderate, intermittent tremor restricted to the head region; 3, visible tremor with occasional quiescent periods affecting the anterior region; 4, continuous tremor, restricted to the extremities and head; 5, continuous, gross, whole body tremor. N=5-6.

Akinesia in BALB/c: Akinesia was measured by noting the latency in seconds (s) of the animals to move all four limbs and the test was terminated if the latency exceeded 180 s. Each animal was initially acclimatized for 5 min on a wooden elevated (30 cm) platform (40 cm×40 cm) used for measuring akinesia in mice. Using a stopwatch, the time taken (s) by the animal to move all the four limbs was recorded. This exercise was repeated five times for each animal. N=5-6.

Catalepsy in BALB/c: The term implies the inability of an animal to correct an externally imposed posture. Catalepsy was measured by placing the animals on a flat horizontal surface with both the hind limbs on a square wooden block (3 cm high) and the latency in seconds was measured to move the hind limbs from the block to the ground. This exercise was repeated five times for each animal. N=5-6.

Swim-test in BALB/c: Swim-test was carried in water tubs (40 cm length×25 cm width×16 cm height). The depth of water was kept at 12 cm and the temperature was maintained at 27±2° C. The animals were wiped dry immediately after the experiment using a dry towel and returned to cages kept at 27±2° C. Swim-score scales were recorded and the following parameters analyzed by an investigator blind to the treatment, with the software The Observer® XT5.0 (Noldus Information Technology, Wageningen, The Netherlands: 0, hind part sinks with head floating; 1, occasional swimming using hind limbs while floating on one side; 2, occasional floating/swimming only; 3, continuous swimming. Swim-test was carried out on different days (3°, 7°, 14° days) after MPTP. N=6.

1. An ethanolic extract of Ptychopetalum olacoides useful for treating or preventing diseases, dysfunctions and disorders of the central nervous system; neurodegenerative disorders and sequel from vascular dementia obtained from a source selected from a group consisting of: stems, barks, leaves, roots and combinations thereof; the extract having antioxidant, anticholine antidepressant and neuroprotective activity.

2. A pharmaceutical composition comprising:
(a) an effective amount of an extract of Ptychopetalum olacoides, and
(b) pharmaceutically acceptable excipients.

3. The pharmaceutical composition according to claim 2, wherein the composition comprises from 0.001% to 99% of extract of Ptychopetalum olacoides.

4. The pharmaceutical composition according to claim 2, wherein the effective amount of the extract of Ptychopetalum olacoides is from about 0.1 mg to about 2000 mg per unit dosage.

5. The pharmaceutical composition according to claim 2, further comprising a component selected from the group consisting of: fixed oils, essential oils, powders, ingredients derived from natural or synthetic sources, vitamins, salts, sugars, and combinations thereof.

6. The pharmaceutical composition according to claim 2, wherein the composition is prepared for topical, oral, inhalable or injectable administration.

7. The pharmaceutical composition according to claim 2, wherein the composition is in a form selected from a group consisting of: tablets, capsules, tinctures, emulsions, water in oil and oil in water liposome creams and gels, microcapsules, nanocapsules, aerosols and pastes.

8. The pharmaceutical composition according to claim 2, wherein the composition is in a form for enteral or parenteral administration, for immediate or modified release

9. The pharmaceutical composition according to claim 2, wherein the composition has antioxidant, anticholine antidepressant and neuroprotective activity.

10. The pharmaceutical composition according to claim 2, wherein the composition promotes brain functions to understand cognitive functions, including: thinking, reasoning, learning, memory, attention span and association.

11. A pharmaceutical compositions useful for treating or preventing diseases, dysfunctions and disorders of the central nervous system; neurodegenerative disorders and sequel from vascular dementia, the compositions comprising:
(a) an effective amount of an extract of Ptychopetalum olacoides, and
(b) pharmaceutically acceptable excipients.

12. The pharmaceutical composition according to claim 11, wherein the diseases, dysfunctions and disorders of the central nervous system is depressed mood or chronic disorders.

13. The pharmaceutical composition according to claim 11, wherein the neurodegenerative disorders is Alzheimer's disease or Parkinson's disease.

14. A method for treating or preventing diseases, dysfunctions and disorders of the central nervous system; neurodegenerative disorders and sequel from vascular dementia in a patient by administering an effective amount of an extract of Ptychopetalum olacoides.

15. The method according to claim 14, wherein the diseases, dysfunctions and disorders of the central nervous system is depressed mood or chronic disorders.

16. The method according to claim 14, wherein the neurodegenerative disorders is depression and related illnesses, Alzheimer's disease or Parkinson's disease.

17. The method according to claim 14, wherein the effective amount of the extract of Ptychopetalum olacoides is from about 0.001 mg/kg/day to about 5000 mg/kg/day, administered in one dose a day or in multiple sub doses a day.

18. The method according to claim 17, wherein the effective amount of the extract of Ptychopetalum olacoides is from about 50 mg/kg/day to about 2000 mg/kg/day, administered in one dose a day or in multiple sub doses a day.
